(19) United States
(12) Patent Application Publication
(10) Pub. No.: US 2010/0022432 A1
(43) Pub. Date: Jan. 28, 2010

(54) USE OF SUPEROXIDE DISMUTASES IN WASHING AND CLEANING AGENTS

(75) Inventors: Karl-Heinz Maurer, Erkrath (DE); Timothy O'Connell, Dusseldorf (DE); Nina Mußmann, Willich (DE); Inken Prüser, Dusseldorf (DE); Ursula Huchel, Koln (DE); Veronika Deppe, Velbert (DE)

Correspondence Address: CONNOLLY BOVE LODGE & HUTZ LLP, PO BOX 2207, WILMINGTON, DE, 19899-2207, US

(73) Assignee: Henkel AG & Co. KGaA, Dusseldorf (DE)

(21) Appl. No.: 12/548,521

(22) Filed: Aug. 27, 2009

Related U.S. Application Data
(63) Continuation-in-part of application No. PCT/EP2008/052334, filed on Feb. 27, 2008.

(30) Foreign Application Priority Data
Mar. 2, 2007 (DE) 102007010785.6

(51) Int. Cl. C12N 9/02; C11D 7/42; C12N 15/53; C12N 15/74; C12N 1/21; C12Q 1/26

(52) U.S. Cl. 510/392; 435/189; 536/23.2; 435/320.1; 435/252.3; 435/25

(57) ABSTRACT

The present invention relates to the use of superoxide dismutases to remove Amadori and Maillard products, and to washing and cleaning agents that contain superoxide dismutases.

### RELATED APPLICATIONS

This application is a continuation-in-part of International Application No. PCT/EP2008/052334, filed Feb. 27, 2008, which is incorporated herein by reference and which claims benefit of German Application No. 102007010785.6, filed Mar. 2, 2007.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is SEQUENCELIST_{—}13744_{—}00084_H07493. The size of the text file is 32.0 kb; the text file was created on Aug. 26, 2009.

The present invention relates to the use of superoxide dismutases for the removal of Amadori and Maillard products, and to washing and cleaning agents that contain superoxide dismutases.

Soiled patches of biological origin, in particular those that derive from foods, are particularly stubborn when they have been heated. Complex reactions occur in this context, and referred to generally by the term “Maillard reaction” or “nonenzymatic browning.” The reducing sugars contained in foods react with amino group of proteins, peptides, or amino acids. This produces the derivative of an N-glycosylamine from which, by Amadori rearrangement, the derivative of a 1-aminodeoxy-2-ketose (“aminoketose”) is formed. A number of further reactions then occur, resulting in e.g. melanoidins (browning), various flavors, and crosslinked proteins.

These Amadori products, or products of the Maillard reaction (Maillard products), are either already contained in the soiled patch on the textile or dish, or are produced only during cleaning and thus may intensify the discoloration; because of the complexity of the reaction product, they are very difficult to remove.

Amadori and Maillard products have furthermore also been described on the hair and the skin. Here as well, these compounds represent undesired products that can be created by oxidative reactions, especially, for example, in the bleaching of hair and in general as the skin and hair age, or in the context of oxidative reactions that occur in patients with insulin-dependent diabetes mellitus (Ahmed, N. (2005) Diabetes Research and Clinical Practice 76, 3-21).

A demand therefore exists for agents that enable the cleavage and therefore also removal of these undesired Amadori and Maillard products, removal of the undesired discolorations also being, above all, the primary focus especially with regard to dishes and textiles.

In this context, amadoriases have already been described in the existing art. These are enzymes that, with the use of oxygen, convert Amadori products into free amino acids with the release of hydrogen peroxide.

It is also known that in nature, Amadori products are broken down in a two-stage process. For this, the Amadori products are first converted, using adenosine triphosphate, by a fructoselysine-6-kinase into a phosphorylated form, which is then broken down by a fructoselysine-6-phosphate deglycanase.

According to the present invention it has now been found, surprisingly, that superoxide dismutases are also capable of breaking down Amadori and Maillard products. The known purpose of superoxide dismutases is actually to convert superoxide (O₂⁻) into hydrogen peroxide with the release of oxygen. The fact that superoxide dismutases might be capable of breaking down Amadori and Maillard products is therefore entirely surprising and could not have been foreseen.

One possible application of superoxide dismutases described in the existing art is, in particular, the breakdown of hydrogen peroxide. DE 4432621, for example, describes a method for bleaching concentrated surfactant solutions in which, in a first step, oxidative bleaching with hydrogen peroxide takes place in the presence of stabilizers, and in a second step, excess unreacted hydrogen peroxide is enzymatically broken down by catalases, glucose oxidases, or superoxide dismutases.

The patent documents DE 2545986, DE 69500016, DE 69027383, and DE 69430326 disclose the use of superoxide dismutases in cosmetic or pharmaceutical agents, but not their use for the cleavage and/or removal of Amadori and/or Maillard products.

A first subject of the present invention is therefore washing and cleaning agents that contain superoxide dismutases.

A further subject of the present invention is therefore likewise the use of superoxide dismutases for the cleavage and/or removal of Amadori and/or Maillard products.

A further subject of the present invention is therefore likewise the use of superoxide dismutases for the removal of soiled patches, in particular stubborn dirt, by preference from hard surfaces and textile fabrics.

A further subject of the present invention is therefore also the use of superoxide dismutases in washing or cleaning agents, in particular for the cleaning of textile fabrics or the cleaning of hard surfaces.

A further subject of the present invention is therefore also the use of superoxide dismutases, if applicable as adjuvants, for the cleaning of textile fabrics and for the cleaning of hard surfaces.

A further subject of the present invention is therefore also the use of superoxide dismutases according to the present invention for the production of a cosmetic or pharmaceutical agent, in particular a topical agent, for the removal of Amadori and/or Maillard products from skin and/hair, and/or for the treatment of age-related discolorations of skin and hair and/or those caused by diabetes mellitus.

The superoxide dismutase to be used according to the present invention is by preference a bacterial enzyme, in particular an enzyme from Bacillus, Oceanobacillus, Bacillus, Streptomyces, Humicola, or Pseudomonas.

In a particularly preferred embodiment, a superoxide dismutase from Bacillus or Oceanobacillus is used.

The enzyme from Bacillus is by preference an enzyme from Bacillus pumilus, Bacillus clausii, Bacillus licheniformis, Bacillus stearothermophilus, Bacillus subtilis, or Bacillus licheniformis.

The enzyme from Oceanobacillus is by preference an enzyme from Oceanobacillus iheyensis.

In a further particularly preferred embodiment, the superoxide dismutase used is one selected from polypeptides from the group of polypeptides having an amino acid sequence according to SEQ ID NO: 2, 4, 6, 8, 10, 12, or 13; naturally occurring or artificially generated mutants, polymorphic forms, or alleles of a polypeptide according to a) having up to 50, 45, 40, or 35, in particular up to 30, 25, 20, or 15, by preference up to 10, 9, 8, 7, 6, 5, 4, 3, or 2 mutations, especially having exactly one mutation; polypeptides having a sequence homology or sequence identity of at least 70, 75, or 80%, particularly preferably at least 82, 84, 86, 88, or 90%, in particular at least 91, 92, 93, 94, or 95%, especially at least 96, 97, 98, or 99%, with an amino acid sequence according to SEQ ID NO: 2, 4, 6, 8, 10, 12, or 13; polypeptides having insertions and/or deletions and/or inversions, in particular exactly one insertion and/or deletion and/or inversion, of up to 50, 45, 40, or 35, in particular up to 30, 25, 20, or 15, by preference up to 10, 9, 8, 7, 6, 5, 4, 3, or 2 amino acids, especially having insertions and/or deletions, in particular exactly one insertion and/or deletion, of exactly one amino acid, with respect to a polypeptide according to a) to c); polypeptides encompassing at least one of the polypeptides recited under a) to d).

In a very particularly preferred embodiment, a superoxide dismutase having an amino acid sequence according to SEQ ID NO: 2, 4, 6, 8, 10, 12, or 13 is used.

Because the superoxide dismutase discovered according to the present invention from Bacillus pumilus is novel, further subjects of the present invention are also polynucleotides selected from: a polynucleotide having a nucleic acid sequence according to SEQ ID NO: 1; a polynucleotide coding for a polypeptide having an amino acid sequence according to SEQ ID NO: 2; naturally occurring or artificially generated mutants or polymorphic forms or alleles of a polynucleotide according to a) having 80, 75, 70, 65, 60, 55, 50, or 45, in particular up to 40, 38, 36, 34, 32, or 30, by preference up to 28, 26, 24, 22, 20, or 18, particularly preferably up to 16, 15, 14, 13, 12, or 11, in particular up to 10, 9, 8, 7, 6, 5, 4, 3, or 2 mutations, especially having exactly one mutation; polynucleotides having a sequence homology or sequence identity of at least 85, 86, or 88%, by preference at least 90, 91, 92, 93, or 94%, especially at least 95, 96, 97, 98, or 99%, with respect to a polynucleotide according to a); polynucleotides hybridizing under stringent conditions with a polynucleotide according to a), “stringent conditions” to be understood by preference as incubation at 60° C. in a solution containing 0.1×SSC and 0.1% sodium dodecylsulfate (SDS), 20×SSC designating a solution containing 3 M sodium chloride and 0.3 M sodium citrate (pH 7.0); polynucleotides having insertions and/or deletions and/or inversions, in particular exactly one insertion and/or deletion and/or inversion, of up to 50, 45, 40, or 35, in particular up to 30, 25, 20, or 15, by preference up to 10, 9, 8, 7, 6, 5, 4, 3, or 2 nucleotides, especially having insertions and/or deletions, in particular exactly one insertion and/or deletion, of exactly one nucleotide, with respect to a polynucleotide according to a) to d); polynucleotides encompassing at least one of the polynucleotides recited under a) to f); polynucleotides complementary to polynucleotides according to a) to g).

The polynucleotides can be present as a single strand or double strand. In addition to the deoxyribonucleic acids, the homologous and complementary ribonucleic acids are also a subject of the invention.

A further subject of the present invention is also vectors, in particular cloning and expression vectors, that contain the polynucleotides according to the present invention, as well as production strains that contain the polynucleotides and/or plasmids according to the present invention.

In a preferred embodiment, the production strain is a Gram-negative bacterium, in particular one of the genera Escherichia coli or Klebsiella, in particular strains of E. coli K12, E. coli B, or Klebsiella planticola, and very particularly derivatives of the strains Escherichia coli BL21 (DE3), E. coli RV308, E. coli DH5α, E. coli JM109, E. coli XL-1, or Klebsiella planticola (Rf).

In a further preferred embodiment, the production strain is a Gram-positive bacterium, in particular one of the genera Bacillus, Oceanobacillus, Staphylococcus, or Cornyebacteria, very particularly of the species B. lentus, B. licheniformis, B. amyloliquefaciens, B. subtilis, B. globigii, B. gibsonii, B. pumilus, or B. alcalophilus, Staphylococcus carnosus, or Cornyebacterium glutamicum.

A further subject of the present invention is also polypeptides selected from polypeptide having an amino acid sequence according to SEQ ID NO: 2; naturally occurring or artificially generated mutants, polymorphic forms or alleles of a polypeptide according to a) having up to 40, 38, 36, 34, 32, or 30, in particular up to 28, 26, 24, 22, 20, or 18, by preference up to 16, 15, 14, 13, 12, or 11, in particular up to 10, 9, 8, 7, 6, 5, 4, 3, or 2 mutations, especially having exactly one mutation; polypeptides that possess a sequence homology or sequence identity of at least 80, 82, 84, 86, or 88%, by preference at least 90, 91, 92, 93, or 94%, especially at least 95, 96, 97, 98, or 99%, with respect to a polypeptide according to a); polypeptides that are coded by a polynucleotide according to the present invention; polypeptides having insertions and/or deletions and/or inversions, in particular exactly one insertion and/or deletion and/or inversion, of up to 50, 45, 40, or 35, in particular up to 30, 25, 20, or 15, by preference up to 10, 9, 8, 7, 6, 5, 4, 3, or 2 amino acids, especially having insertions and/or deletions, in particular exactly one insertion and/or deletion, of exactly one amino acid, with respect to a polypeptide according to a) to c); polypeptides encompassing at least one of the polypeptides recited under a) to e).

The polypeptide in this context is by preference a superoxide dismutase, in particular a superoxide dismutase from bacteria, by preference from Bacillus, particularly preferably from Bacillus pumilus.

A further subject of the present invention is also a method for identifying superoxide dismutases that break down Amadori and/or Maillard products, encompassing the following steps:

a protein-containing sample is provided;
Amadori and/or Maillard products, as well as a reagent for detecting hydrogen peroxide, are brought into contact with the protein-containing sample;
based on the reagent for detecting hydrogen-peroxide, the presence of hydrogen peroxide, and thus the enzymatic breakdown of Amadori and/or Maillard products, is detected.

The washing and cleaning agents according to the present invention can be all conceivable types of cleaning agents, both concentrates and agents to be utilized undiluted, for use on a commercial scale, in a washing machine or for hand laundering or cleaning. These include, for example, washing agents for textiles, carpets, or natural fibers, for which the term “washing agent” is used in accordance with the present invention. These also include, for example, dishwashing agents for automatic dishwashers, or manual dishwashing agents or cleaners for hard surfaces such as metal, glass, porcelain, ceramic, tiles, stone, painted surfaces, plastics, wood, or leather; for these, the term “cleaning agent” is used in accordance with the present invention. Sterilizing and disinfecting agents are also to be regarded in the broader sense, in accordance with the present invention, as washing and cleaning agents.

Embodiments of the present invention encompass all presentation forms established according to the existing art, and/or all appropriate presentation forms, of the washing or cleaning agents according to the present invention. Included among these are, for example, solid, powdered, liquid, gelled, or pasty agents, optionally also made up of multiple phases, compressed or uncompressed; also included thereamong are, for example, extrudates, granulates, tablets, or pouches, both in large containers and packaged in single portions.

In a preferred embodiment, the washing or cleaning agents according to the present invention contain superoxide dismutases in a quantity from 2 μg to 20 mg, by preference from 5 μg to 17.5 mg, particularly preferably from 20 μg to 15 mg, very particularly preferably from 50 μg to 10 mg, per gram of the agent. All integral and nonintegral values lying respectively between these numbers are included.

In addition to a polypeptide according to the present invention, a washing or cleaning agent according to the present invention optionally contains further ingredients, in particular further enzymes, enzyme stabilizers, surfactants, e.g. nonionic, anionic, and/or amphoteric surfactants, bleaching agents, builders, polymers, bleach activators, bleach catalysts solvents, thickeners, sequestering agents, electrolytes, optical brighteners, graying inhibitors, color transfer inhibitors, glass corrosion inhibitors, corrosion inhibitors, disintegration adjuvants, foam inhibitors, abrasives, dyes, fragrances, antimicrobial active substances, UV absorbents, crease prevention agents, antistatic agents, soil-release active substances or soil repellents, propellants, or perfume carriers. These and further preferred ingredients are described in more detail below.

With regard to detergency builders, surfactants, polymers, bleaching agents, bleach activators, bleach catalysts, solvents, thickeners, optical brighteners, graying inhibitors, crease prevention agents, antistatics, glass corrosion inhibitors, corrosion inhibitors, soil repellents, color transfer inhibitors, foam inhibitors, abrasives, disintegration agents or adjuvants, acidifying agents, dyes, fragrances, antimicrobial active substances, UV absorbents, and propellants usable by preference according to the present invention, and preferred utilization quantities thereof, reference is made to International Application WO2008/107346.

The active oxygen content of the washing or cleaning agents, in particular of the automatic dishwashing agents, is, based in each case on the total weight of the agent, by preference between 0.4 and 10 wt %, particularly preferably between 0.5 and 8 wt %, and in particular between 0.6 and 5 wt %. Particularly preferred agents have an active oxygen content above 0.3 wt %, preferably above 0.7 wt %, particularly preferably above 0.8 wt %, and in particular above 1.0 wt %.

In order to increase the washing or cleaning performance, respectively, of washing or cleaning agents, in addition to the superoxide dismutase to be used according to the present invention it is also possible to use further enzymes. Included among these are, in particular, proteases, amylases, lipases, hemicellulases, cellulases, amadoriases, perhydrolases, or oxidoreductases, as well as preferably mixtures thereof. Particularly preferably, the further enzyme to be used is selected from proteases, amylases, and amadoriases. These enzymes are by preference of natural origin; proceeding from the natural molecules, improved variants are available for use in washing or cleaning agents, which variants are used in correspondingly preferred fashion. Washing or cleaning agents contain enzymes by preference in total quantities from 1×10⁻⁶ to 5 wt %, based on active protein. The protein concentration can be determined with the aid of known methods, for example the BCA method or the biuret method.

With regard to further enzymes usable by preference according to the present invention, and enzyme stabilizers usable by preference, and to their preferred forms of administration and utilization quantities, reference is made to International Application WO2008/107346.

A separate subject of the invention is represented by methods for automatic cleaning of textiles or hard surfaces in which a superoxide dismutase is used in at least one of the method steps.

This embraces both manual and automatic methods, automatic methods being preferred because they are more precisely controllable with regard, for example, to quantities used and contact times.

Methods for cleaning textiles are generally notable for the fact that in multiple method steps, a variety of substances having cleaning activity are applied onto the material to be cleaned, and are washed off after the contact time, or that the material to be cleaned is otherwise treated with a washing agent or a solution of said agent. The same applies to methods for cleaning all materials other than textiles, which are grouped under the term “hard surfaces.” All conceivable washing or cleaning methods can supplemented in at least one of the method steps with superoxide dismutases, and then represent embodiments of the present invention.

A separate subject of the invention is represented by the use of superoxide dismutases to clean textiles or hard surfaces.

The concentration ranges listed above apply in correspondingly preferred fashion to these aforesaid uses.

This is because superoxide dismutases can be used, in particular in accordance with the properties and methods described above, to eliminate soiled patches from textiles or from hard surfaces. Embodiments are represented, for example, by hand laundering, manual removal of spots from textiles or from hard surfaces, or use in conjunction with an automatic method.

In a preferred embodiment of this use, the superoxide dismutases are provided in the context of one of the aforementioned formulations for agents according to the present invention, by preference washing and cleaning agents.

A further subject of the present invention is also a product containing a composition according to the present invention or a washing or cleaning agent according to the present invention, in particular a cleaner according to the present invention for hard surfaces, and a spray dispenser. The product can in this context be both a single-chamber and a multi-chamber vessel, in particular a two-chamber vessel. In this context, the spray dispenser is preferably a manually activated spray dispenser, selected in particular from the group encompassing aerosol spray dispensers (pressurized-gas containers, also referred to inter alia as a spray can), spray dispensers that themselves build up pressure, pump spray dispensers, and trigger spray dispensers, in particular pump spray dispensers and trigger spray dispensers having a container made of transparent polyethylene or polyethylene terephthalate. Spray dispensers are described more exhaustively in WO 96/04940 (Procter & Gamble) and in the U.S. patents cited therein regarding spray dispensers, to which patents in their entirety reference is made in this regard, and the content of which is hereby incorporated into this application. Trigger spray dispensers and pump atomizers possess the advantage, as compared with pressurized-gas containers, that no propellant needs to be used. By means of suitable particle-capable attachments, nozzles, etc. (so-called “nozzle valves”) on the spray dispenser, in this embodiment an enzyme that may be contained can optionally also be added to the agent in a form immobilized on particles, and thus metered as a cleaning foam.

Automatic dishwashing agents that are particularly preferred according to the present invention encompass 5 to 70 wt %, by preference 10 to 60 wt %, and in particular 20 to 50 wt % detergency builder(s), with the exception of polymers having washing and cleaning activity, by preference phosphates; 2 to 28 wt %, by preference 4 to 20 wt %, and in particular 6 to 15 wt % polymers having washing and cleaning activity; 0.5 to 10 wt %, by preference 1 to 8 wt %, and in particular 2 to 6 wt % surfactant(s), by preference nonionic and/or amphoteric surfactant(s), particularly preferably nonionic surfactant(s); 0.5 to 8 wt %, by preference 1 to 7 wt %, and in particular 2 to 6 wt % superoxide dismutases, if applicable in combination with amylases, proteases, and/or amadoriases; 2 to 20 wt %, by preference 4 to 15 wt %, and in particular 6 to 12 wt % bleaching agent, by preference percarbonate; and 0.01 to 5 wt %, by preference 0.02 to 4 wt %, and in particular 0.05 to 3 wt % bleach catalysts.

Very particularly preferred automatic dishwashing agents encompass

5 to 70 wt %, by preference 10 to 60 wt %, and in particular 20 to 50 wt % phosphates;
2 to 28 wt %, by preference 4 to 20 wt %, and in particular 6 to 15 wt % polymers having washing and cleaning activity;
0.5 to 10 wt %, by preference 1 to 8 wt %, and in particular 2 to 6 wt % nonionic surfactant(s);
0.5 to 8 wt %, by preference 1 to 7 wt %, and in particular 2 to 6 wt % superoxide dismutases, it applicable in combination with amylases, proteases, and/or amadoriases;
2 to 20 wt %, by preference 4 to 15 wt %, and in particular 6 to 12 wt % percarbonate; and
0.01 to 5 wt %, by preference 0.02 to 4 wt %, and in particular 0.05 to 3 wt % bleach catalysts.

Automatic dishwashing agents according to the present invention can be prepared in various ways. The agents according to the present invention can exist in solid or liquid presentation forms, and as a combination of solid and liquid presentation forms.

Powders, granulates, extrudates, or compactates, in particular tablets, are suitable in particular as solid presentation forms. The liquid presentation forms, based on water and/or organic solvents, can be present in thickened fashion in the form of gels.

Agents according to the present invention can be prepared in the form of single-phase or multi-phase products. Automatic dishwashing agents having one, two, three, or four phases are particularly preferred. Automatic dishwashing agents characterized in that it is present in the form of a prefabricated dispensing unit having two or more phases, are particularly preferred.

The individual phases of multi-phase agents can have the same or different aggregate states. Automatic dishwashing agents that comprise at least two different solid phases and/or at least two liquid phases and/or at least one solid and at least one solid phase are preferred in particular.

Automatic dishwashing agents according to the present invention are preferably pre-packaged into dispensing units. These dispensing units preferably encompass the quantity of substances having washing or cleaning activity that is necessary for one cleaning cycle. Preferred dispensing units have a weight between 12 and 30 g, preferably between 14 and 26 g, and in particular between 16 and 22 g.

With particular preference, the volume of the aforesaid dispensing units, and their three-dimensional shape, are selected so that dispensability of the pre-packaged units via the dispensing chamber of an automatic dishwasher is guaranteed. The volume of the dispensing unit is therefore preferably between 10 and 35 ml, by preference between 12 and 30 ml, and in particular between 15 and 25 ml.

The automatic dishwashing agents according to the present invention, in particular the prefabricated dispensing agents, particularly preferably possess a water-soluble casing.

The cosmetic and pharmaceutical agents according to the present invention are preferably those for topical application. “Topical” cosmetic or pharmaceutical agents are to be understood, in particular, as all types of cleaning and care-providing agents for human skin or human hair, in particular cleaning agents.

Examples that may be recited of topical cosmetic or pharmaceutical agents according to the present invention are liquid soaps, lotions, sprays, creams, gels, emulsions, cleaning liquids, cleaning milks, deodorants, antiperspirants, salves, hair therapies, shampoos, peels, and oral and dental care agents. These agents can contain further constituents that are usual for topical cosmetic or pharmaceutical agents, in this case including especially constituents such as those recited above for washing and cleaning agents, wherein the further constituents can be selected, for example, from surfactants, further enzymes, light protection agents, plant extracts, vitamins, protein hydrolysates, saccharides, polymers, fatty substances, thickening agents, perfume oils, solvents, and luster agents.

The exemplifying embodiments that follow explain the invention further without limiting it thereto.

### EXAMPLES

### Example 1

### Identification of Suitable Superoxide Dismutases (SODS) by Native Polyacrylamide Gel Electrophoresis

After standard culturing in LB medium that contains the crude substrate, the cells are harvested and digested. The crude extract has a native sample buffer added to it, and is separated on a 10% Tris-glycine gel using Tris-glycine running buffer.

Each sample is applied twice. Half the gel is stained with Coomassie according to a standard protocol, the other half is placed onto an activity plate. This activity plate contains the substrate and an H₂O₂ detection system, so that the SOD band produces a blue coloration.

To produce the activity plate, 1 g agar was boiled in 50 ml 120 mM Tris-HCl buffer (pH 8.5). In a second solution, 0.5 ml crude substrate (50 g crude substrate/100 ml), 26.8 mg 4-chloronaphthol, and horseradish peroxidase (2 ml of a 54 U/ml solution) were dissolved in 50 ml double-distilled water. After purification of the solutions, the agar plates were cast and left to cool.

The substrate, a mixture of Maillard and Amadori products, was synthesized from N-ε-acetyl-L-lysine and glucose for the assay and the medium. N-a-fructosyl-N-ε-acetyllysine was synthesized using the method of Finot, P. A. and Mauron, J., (1969) Helv. Chim. Acta 52, 1488-1495, except that chromatography was not carried out, so that a mixture of Maillard and Amadori products is obtained.

Once suitable proteins have been identified, they can be terminally sequenced in order to obtain suitable primers for amplification of the protein DNA.

1. A washing or cleaning agent containing a superoxide dismutase.

2. The washing or cleaning agent of claim 1, wherein the superoxide dismutase is a bacterial enzyme.

3. The washing or cleaning agent of claim 2, wherein the bacterial enzyme is from the genera Bacillus or Oceanobacillus.

4. The washing or cleaning agent of claim 1, wherein the enzyme is a polypeptide selected from the group consisting of:
a) polypeptides having the amino acid sequence of SEQ ID NO: 2, 4, 6, 8, 10, 12, or 13;
b) naturally occurring or artificially generated mutants, polymorphic forms, or alleles of the polypeptides of a) having up to 50 mutations;
c) polypeptides having sequence homology or sequence identity of at least 70% with the amino acid sequences of SEQ ID NO: 2, 4, 6, 8, 10, 12, or 13;
d) polypeptides having insertions and/or deletions and/or inversions of up to 50 amino acids with respect to the polypeptides of a) to c); and
e) polypeptides encompassing at least one of the polypeptides of a) to d).

5. The washing or cleaning agent of claim 1, further comprising at least one active substance selected from the group consisting of a surfactant; an enzyme selected from proteases, amylases, and amadoriases; and enzyme stabilizers, bleaching agents, builders, polymers, bleach activators, glass corrosion inhibitors, corrosion inhibitors, disintegration adjuvants, fragrances, and perfume carriers.

6. The washing or cleaning agent of claim 1, wherein the washing or cleaning agent is selected from the group consisting of automatic dishwashing agents, manual dishwashing agents, household cleaners, toilet cleaners, and powdered and liquid textile washing agents.

7. A method for cleaving and/or removing Amadori and/or Maillard products, the method comprising contacting the products with the washing or cleaning agent of claim 1.

8. A method for removing a soiled patch, the method comprising contacting the patch with the washing or cleaning agent of claim 1.

9. The method of claim 8, wherein the soiled patch is stubborn dirt.

10. A method for cleaning a textile fabric or a hard surface, the method comprising contacting the fabric or surface with the washing or cleaning agent of claim 1.

11. The method of claim 10, wherein the superoxide dismutase is an adjuvant.

12. A method for producing a cosmetic or pharmaceutical agent for the removal of Amadori and/or Maillard products from skin and hair, and/or for the treatment of age-related discolorations of skin and hair, and/or the treatment of discolorations of skin and hair caused by diabetes mellitus, the method comprising incorporating a superoxide dismutase into the agent.

13. The method of claim 12, wherein the cosmetic or pharmaceutical agent is a topical agent.

14. A polynucleotide selected from the group consisting of:
a) a polynucleotide having the nucleic acid sequence of SEQ ID NO: 1;
b) a polynucleotide coding for a polypeptide having the amino acid sequence of SEQ ID NO: 2;
c) naturally occurring or artificially generated mutants or polymorphic forms or alleles of the polynucleotide of a) having up to 80 mutations;
d) polynucleotides having a sequence homology or sequence identity of at least 85% with respect to the polynucleotide of a);
e) polynucleotides hybridizing under stringent conditions with the polynucleotide of a);
f) polynucleotides having deletions and/or insertions and/or inversions of up to 50 nucleotides with respect to the polynucleotide of a);
g) polynucleotides encompassing at least one of the polynucleotides recited under a) to f);
h) polynucleotides complementary to the polynucleotides of a) to g).

15. A vector comprising the selected polynucleotide of claim 14.

16. A host cell comprising the selected polynucleotide of claim 14.

17. A host cell comprising the vector of claim 15.

18. A polypeptide selected from the group consisting of:
a) a polypeptide having the amino acid sequence of SEQ ID NO: 2;
b) naturally occurring or artificially generated mutants, polymorphic forms, or alleles of the polypeptide of a) having up to 40 mutations;
c) polypeptides that possess a sequence homology or sequence identity of at least 80% with respect to the polypeptide of a);
d) polypeptides having insertions and/or deletions and/or inversions of up to 50 amino acids with respect to the polypeptides of a) to c);
e) polypeptides encompassing at least one of the polypeptides recited under a) to d).

19. A method for identifying superoxide dismutases that break down Amadori and/or Maillard products, the method comprising the steps of
a) providing a protein-containing sample;
b) contacting the protein-containing sample with Amadori and/or Maillard products and a reagent for detecting hydrogen peroxide;
c) detecting the presence of hydrogen peroxide, and thus the enzymatic breakdown of Amadori and/or Maillard products, based on the reagent for detecting hydrogen peroxide.


SEQUENCE LISTING (13 sequences)

SEQ ID NO: 1 - DNA, 609 bp, Bacillus pumilus
  CDS (1)..(609)
atggcttacaaacttccagaattaccatatgcttacgatgcattagaaccacatatcgacaaggaaacaatgacgattcaccacacaaaacaccacaacacatacgtaacaaacttaaacaaagcaatcgaaggtgtatcagctcttgaagatcaatcaatcgaagagctagtggctaacctaaactctgtgcctgagaacatccgtacagctgtacgtaacaatggcggaggacatgctaaccactcattattctggacgcttctttcaccaaacggtggcggcgcaccaactggtgaacttgcagatgcaatcgaaaaagaattaggcggatttgaaaaattcaaatctgatttcgctgcagcagctgcaggacgctttggttctggttgggcatggcttgttgtgaacaatggcaaacttgaaatcacaagcacgccaaaccaagattctcctttaactgaaggaaaaacaccaattctaggacttgacgtttgggagcatgcttactacctaaactaccaaaaccgtcgtcctgattacatttcagcattctggaatgttgtgaactgggatgaagtggcacgcctttacagcgaagcaaaataa

SEQ ID NO: 2 - protein, 202 aa, Bacillus pumilus
MAYKLPELPYAYDALEPHIDKETMTIHHTKHHNTYVTNLNKAIEGVSALEDQSIEELVANLNSVPENIRTAVRNNGGGHANHSLFWTLLSPNGGGAPTGELADAIEKELGGFEKFKSDFAAAAAGRFGSGWAWLVVNNGKLEITSTPNQDSPLTEGKTPILGLDVWEHAYYLNYQNRRPDYISAFWNVVNWDEVARLYSEAK

SEQ ID NO: 3 - DNA, 609 bp, Bacillus clausii KSM-K16
  CDS (1)..(609)
atggcttacaaactaccagaattgccttatgctgcaaatgcacttgagccgcatattgatgaggcgacaatgaacattcaccacggcaaacaccacaatacgtatgtaacaaacttaaatgctgcattggaagggcatgcagcccttgcagagaagagcattgaagacttggtggcaaacttggatgctgttccagaaaacattcgtacagcggttcgcaacaatggcggaggccatgcaaaccacacatttttttggcaaattttaagtccaaatggtggcggcgagccgactggtgctctagcagaagatattaaatcaactttcggcagcgttgaagaattcaaaaacaaatttgcagacgcagcaaaagggcgctttggttcaggatgggcttggctagttgtcaacaacggcaatttagaaattacaagcacgcctaaccaagatacacctttatctgaagggaaaacgcctatcctcggacttgacgtctgggaacacgcttactacttgaactaccaaaaccgtcgcccagactacattgcagctttctggaatgtcgtcaattgggacgaggttagcaaacgttacgaagcagcaaaataa

SEQ ID NO: 4 - protein, 202 aa, Bacillus clausii KSM-K16
MAYKLPELPYAANALEPHIDEATMNIHHGKHHNTYVTNLNAALEGHAALAEKSIEDLVANLDAVPENIRTAVRNNGGGHANHTFFWQILSPNGGGEPTGALAEDIKSTFGSVEEFKNKFADAAKGRFGSGWAWLVVNNGNLEITSTPNQDTPLSEGKTPILGLDVWEHAYYLNYQNRRPDYIAAFWNVVNWDEVSKRYEAAK

SEQ ID NO: 5 - DNA, 609 bp, Bacillus licheniformis DSM 13
  CDS (1)..(609)
atggcttacaaacttccagaattaccttatgcttacgatgcgttagaacctcacatcgacaaagagacgatgaatattcaccatacgaagcaccataacacatatgttacaaaattgaacgaagcggtggcaggcaaacaggatcttgaaagcaaatctgttgaagagcttgttgcaaaccttgatgcagtaccggaaaacatccgtacagctgtccgcaacaatggcggagggcatgccaaccactcattgttctggaaacttctttctccaaacggaggaggcgctccaactggtgaattggcagaagccatcaacagcaaattcggcagcttcgatcaattcaaagaagactttgcggctgcagcagcaggccgtttcggctccggctgggcatggcttgttgtcaataacggagagcttgaaatcacaagcacgccaaaccaagattctcctctttcagaaggcaagactcctatcttaggtcttgatgtatgggagcacgcatactacctgaattatcaaaaccgccgtccagattacattaaagcattctggaacgtcgttaactgggatgaagttgcacgcctttacagcgaagcaaaataa

SEQ ID NO: 6 - protein, 202 aa, Bacillus licheniformis DSM 13
MAYKLPELPYAYDALEPHIDKETMNIHHTKHHNTYVTKLNEAVAGKQDLESKSVEELVANLDAVPENIRTAVRNNGGGHANHSLFWKLLSPNGGGAPTGELAEAINSKFGSFDQFKEDFAAAAAGRFGSGWAWLVVNNGELEITSTPNQDSPLSEGKTPILGLDVWEHAYYLNYQNRRPDYIKAFWNVVNWDEVARLYSEAK

SEQ ID NO: 7 - DNA, 615 bp, Bacillus stearothermophilus
  CDS (1)..(615)
atgccatttgaattgccagcattgccgtatccgtatgatgctctggagccgcacatcgacaaagaaacgatgaacattcaccacacgaagcaccataacacatacgttacaaatttgaatgcggcgcttgaaggacatccggatttgcaaaacaaatcgctcgaagaactgctcagcaatttggaagcccttccggaaagcatccgcacggcggtgcgcaacaacggcggcggccatgcgaaccactcgcttttctggacgattttgtcgccaaatggcggcggcgagccgacgggtgagctggctgacgccatcaacaaaaaattcggcagcttcaccgcgttcaaagacgagttttcgaaagcagcggccggccgtttcggttccggttgggcatggcttgttgtgaacaacggcgagctggaaatcacaagcacgccgaaccaagattcgccgattatggaaggcaaaacgccgattctcggcttggacgtttgggagcatgcgtactacttgaaataccaaaaccgccgtccggaatacattgccgcattctggaacgtcgtcaactgggacgaagtggcgaaacggtacagcgaagcgaaagcaaaataa

SEQ ID NO: 8 - protein, 204 aa, Bacillus stearothermophilus
MPFELPALPYPYDALEPHIDKETMNIHHTKHHNTYVTNLNAALEGHPDLQNKSLEELLSNLEALPESIRTAVRNNGGGHANHSLFWTILSPNGGGEPTGELADAINKKFGSFTAFKDEFSKAAAGRFGSGWAWLVVNNGELEITSTPNQDSPIMEGKTPILGLDVWEHAYYLKYQNRRPEYIAAFWNVVNWDEVAKRYSEAKAK

SEQ ID NO: 9 - DNA, 615 bp, Bacillus thermoleovorans
  CDS (1)..(615)
atgccatttgaattgccagcattgccgtatccgtatgatgcgcttgagccgcacatcgacaaagaaacgatgaacattcaccacacgaagcaccataacacatacgttacaaatttgaatgcggcgcttgaagggcatccggatttgcaaaacaaatcgctcgaagaattgctcagcaatttggaagcccttccggaaagcattcgcacggcggtgcgcaacaacggcggcggtcatgcaaaccactcgcttttctggacgattttgtcgccaaatggcggcggtgagccgacgggtgagctggctgaggcgatcaacaaaaaattcggcagcttcaccgcgtttaaagacgagttttcgaaagcagcggctggccgtttcggttctggctgggcatggcttgtcgtgaacaacggcgagctggaaattacgagcacgccgaaccaagactcgccgatcatggaaggcaaaacgccgattctcggcttggacgtttgggagcatgcgtactacttgaaataccaaaaccgccgtccggaatacattgccgcattctggaacattgtcaactgggacgaagtggcgaaacggtacagcgaagcgaaagcgaagtaa

SEQ ID NO: 10 - protein, 204 aa, Bacillus thermoleovorans
MPFELPALPYPYDALEPHIDKETMNIHHTKHHNTYVTNLNAALEGHPDLQNKSLEELLSNLEALPESIRTAVRNNGGGHANHSLFWTILSPNGGGEPTGELAEAINKKFGSFTAFKDEFSKAAAGRFGSGWAWLVVNNGELEITSTPNQDSPIMEGKTPILGLDVWEHAYYLKYQNRRPEYIAAFWNIVNWDEVAKRYSEAKAK

SEQ ID NO: 11 - DNA, 612 bp, Oceanobacillus iheyensis HTE831
  CDS (1)..(612)
atggcaaaatttgaattaccggaattaccatatgcatatgatgcattggaaccgacaattgacaaagaaacaatgaacattcatcatacgaaacatcacaacacgtatgtaactaaattaaatgatgcattggaaggacatgcagaccttcaaagtaagtctgtagaagaattaattagtaatcttgatgcagtccctgaaaatgcaaaaactgcagtacgtaacaatggtggcggacatgctaaccatagcctattttggaagttattatcaccaaatggcggcggtgagccaacaggtgaattagctgataaaattaatgctaaatttggttcattagacaaattcaaagaagaatttgcagcagctgcagcaggacgtttcggttctggatgggcttggctaattgttaacaatggtgaactagaaattacaagcacaccaaaccaagattcaccattaatggaaggaaaaactcctgtattaggacttgatgtttgggaacacgcttattatcttaaatatcaaaacaaacgcccagagtacatttcagcattctggaatgtagttaattgggatcaagttgcaaaaaactacgaagaagctaagtaa

SEQ ID NO: 12 - protein, 203 aa, Oceanobacillus iheyensis HTE831
MAKFELPELPYAYDALEPTIDKETMNIHHTKHHNTYVTKLNDALEGHADLQSKSVEELISNLDAVPENAKTAVRNNGGGHANHSLFWKLLSPNGGGEPTGELADKINAKFGSLDKFKEEFAAAAAGRFGSGWAWLIVNNGELEITSTPNQDSPLMEGKTPVLGLDVWEHAYYLKYQNKRPEYISAFWNVVNWDQVAKNYEEAK

SEQ ID NO: 13 - protein, 202 aa, Bacillus subtilis subsp. subtilis str. 168
MAYELPELPYAYDALEPHIDKETMTIHHTKHHNTYVTNLNKAVEGNTALANKSVEELVADLDSVPENIRTAVRNNGGGHANHKLFWTLLSPNGGGEPTGALAEEINSVFGSFDKFKEQFAAAAAGRFGSGWAWLVVNNGKLEITSTPNQDSPLSEGKTPILGLDVWEHAYYLNYQNRRPDYISAFWNVVNWDEVARLYSERK